(19) United States
(12) Patent Application Publication
(10) Pub. No.: US 2025/0382666 A1
(43) Pub. Date: Dec. 18, 2025

(54) ASSAY DEVICE FOR ISOTHERMAL AMPLIFICATION AND DETECTION OF NUCLEIC ACIDS

(71) Applicant: DOMUS DIAGNOSTICS, INC., Park City, UT (US)

(72) Inventors: Joshua R. BUSER, Park City, UT (US); John H. REIF, Durham, NC (US); Xin SONG, Raleigh, NC (US); Dan MORGAN, Park City, UT (US); Josh BECKMANN, Park City, UT (US); Jacquelyn M. WALEJKO, Durham, NC (US); Paul CHAPMAN, Park City, UT (US)

(21) Appl. No.: 19/239,464

(22) Filed: Jun. 16, 2025

Related U.S. Application Data
(60) Provisional application No. 63/660,882, filed on Jun. 17, 2024.

(51) Int. Cl. C12Q 1/6844; B01L 3/00; C12Q 1/70

(52) U.S. Cl. CPC C12Q 1/6844; B01L 3/5023; B01L 3/502715; C12Q 1/701; B01L 2200/143; B01L 2200/16; B01L 2300/0864; B01L 2300/1877

(57) ABSTRACT

Disclosed is an assay device for isothermal amplification and detection of one or more target nucleic acids. The assay device can utilize a simple mechanical design, an effective reagent chemistry, and an electricity-free heating configuration that enable a low-cost, highly accessible assay device. The assay device can be utilized in the field and/or in point-of-care (POC) applications without the need for sophisticated laboratory equipment, without the need for electricity or batteries, and without significantly sacrificing assay performance relative to conventional, higher-cost laboratory approaches.

### CROSS-REFERENCE TO RELATED APPLICATIONS

This application claims priority to and the benefit of U.S. Provisional Application No. 63/660,882, filed Jun. 17, 2024, the entirety of which is incorporated herein by reference.

### BACKGROUND

There is growing demand for diagnostic tools that can provide rapid and specific testing without requiring a patient to go to a hospital or wait several weeks for a lab result. The COVID 19 (SARS-CoV-2) pandemic, for example, highlighted the need for a shift in testing for infectious diseases. Portable nucleic acid testing has proven to be an essential tool in evaluating both symptomatic and asymptomatic subjects, helping them and their healthcare providers to provide time-critical plans of action to best address their health and the health of those around them.

Portable assays relying on nucleic acid amplification require the use of reagents to provide the required primers, enzymes, buffers, and deoxynucleotide triphosphates (dNTPs), for example. Conventional laboratory-based assays, such as those based on conventional polymerase chain reaction (PCR), require cold storage and/or cold chain transport logistics, which can increase the overall complexity and cost of the assay. Recent years have witnessed a dramatic increase in the research and development of alternative nucleic acid detection techniques that are simple, low-cost, and better suited for point-of-care (POC) and at-home diagnostic applications without reliance on specialized instrumentation and trained personnel.

Although rapid antigen tests are often available for the diagnosis of infectious diseases, these tests lack the sensitivity and accuracy of molecular techniques based on amplification of target nucleic acids. One example amplification protocol is loop-mediated isothermal amplification (LAMP, or RT-LAMP if the target is RNA), which uses a set of four to six primers and a strand-displacing DNA polymerase (or in addition, a reverse transcriptase if the target is RNA) to exponentially amplify trace amount of the target nucleic acid under isothermal conditions (i.e., reactions take place at a constant temperature without the need for thermocycling). However, despite being more tolerant to inhibitors than the conventional PCR, the performance of isothermal amplification techniques such as LAMP and RT-LAMP still largely depends on the quality of samples, and conventional approaches typically require dedicated pre-processing via a combination of thermal, mechanical, and/or chemical treatments for sample inactivation, lysis, nucleases inhibition, nucleic acid extraction, and purification in separate steps before the sample is prepared for a downstream amplification reaction.

Accordingly, there is an ongoing need for assay devices that promote broad accessibility and that can effectively provide diagnostic results without requiring complicated, expensive laboratory equipment.

### SUMMARY

The present disclosure is directed to an assay device for isothermal amplification and detection of one or more target nucleic acids. Embodiments of the assay device disclosed herein can incorporate a simple mechanical design, an effective reagent chemistry, and an electricity-free heating configuration that enable a low-cost assay device that does not require specialized equipment or highly trained personnel. The assay device can be utilized in the field and/or in POC applications without the need for sophisticated laboratory equipment, without the need for electricity or batteries, and without significantly sacrificing assay performance relative to conventional, higher-cost laboratory approaches.

The assay device can include a reaction card with a laminate construction that is straightforward to manufacture. The design omits complicated valves, hinges, and other moving parts, and utilizes a simple microfluidics design that can automatically transfer a collected sample and associated buffer to dedicated reaction chambers where sample processing (including sample inactivation, sample lysis, nuclease inhibition, nucleic acid extraction, nucleic acid stabilization) and nucleic acid amplification (e.g., via LAMP or RT-LAMP) take place. A readout indicator included in the reaction enables direct visualization of the assay results.

The assay device beneficially enables simple and easy operation that (i) processes the sample, (ii) releases and amplifies target nucleic acids, and (iii) activates a readout indicator, all without requiring complicated processing steps, sophisticated equipment, or even electricity or batteries.

The disclosed embodiments may be particularly useful with portable assay devices designed for use outside of a laboratory setting, such as assays designed for remote or field use where power and/or standard laboratory equipment are not readily available or not economically feasible. Examples also include assays designed for testing at a POC setting, home setting, a temporary “pop-up” clinic setting, a mobile medical unit, or even a healthcare facility where cost or operator training limitations reduce the feasibility of providing conventional laboratory-grade assays.

In one embodiment, the assay device comprises a buffer tube containing a sample processing buffer formulated for mixing with a sample, and a reaction card configured to receive the buffer tube and at least a portion of its contents. The reaction card comprises a receiving area for receiving the contents of the buffer tube, one or more reaction chambers each containing a master mix formulated to enable amplification of a target nucleic acid, and one or more channels fluidically connecting the receiving area with the one or more reaction chambers and configured to deliver the received contents of the buffer tube to the one or more reaction chambers. The one or more reaction chambers are visible through the reaction card such that a readout indicator within each reaction chamber is visible for indicating results of the assay.

In one embodiment, a method for assaying a sample for one or more target nucleic acids comprises: adding a sample to a sample processing buffer within a buffer tube of an assay device to form a sample mixture; connecting the buffer tube to a reaction card of the assay device to cause at least a portion of the sample mixture to migrate to one or more reaction chambers; and providing heat to the reaction card to drive isothermal amplification of target nucleic acids if such are present within the sample.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the disclosure will become apparent and more readily appreciated from the following description, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1A is a perspective view of an example assay device;

FIG. 1B illustrates an example readout card of the assay device, the readout card allowing direct visualization of the reaction chambers to enable ready determination of assay results;

FIG. 2A is an exploded view of a reaction card configured to receive a sample mixture from a buffer tube and to direct the sample mixture to the reaction chambers where amplification and detection reactions can occur;

FIG. 2B is another view of the card top portion of the reaction card to better illustrate the reaction chambers, microfluidic channels, and receiving area where the sample mixture is received;

FIGS. 3A through 3D illustrate more detailed views of different example reaction chambers;

FIGS. 4A through 4C illustrate an example workflow for initiating a test with the assay device;

FIGS. 5A through 5F illustrate a process for assembling an assay device, including assembling the outer cover and internal components such as the reaction card, heat source, and insulation layers;

FIGS. 6A and 6B illustrates cross-sectional side views of the assay device, showing an example heating/insulation configuration that includes a temperature regulator, heat source, and an air inlet that function together to enable sustained and stable delivery of heat to the reaction card to drive the desired isothermal reactions;

FIG. 7 is a graph illustrating results of heat profile testing showing that the assay device can maintain a stable temperature within target range at the reaction card for over an hour;

FIG. 8 illustrates temperature profiles for assay devices tested at 15° C. ambient environment and 30° C. ambient environment;

FIGS. 9A and 9B illustrate the example reaction card after an in-device lyophilization process in exploded view and intact perspective view, respectively.

### DETAILED DESCRIPTION

### Structural & Microfluidic Features

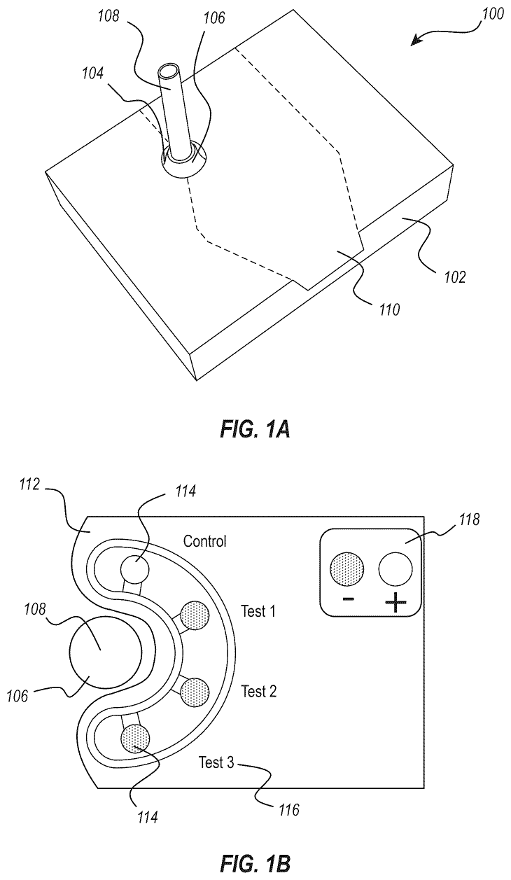

FIG. 1A illustrates an example assay device 100. As shown, the assay device 100 includes an outer cover 102 that houses internal components of the device, including a microfluidic reaction card, insulation, heat source, and temperature regulator. These internal components are shown and described in greater detail below. The outer cover 102 includes an aperture 104 through which a receiving base 106 is exposed. The receiving base 106 is configured to receive and engage with a buffer tube 108 and to fluidically connect the buffer tube 108 to fluid channels of the reaction card.

The buffer tube 108 is configured to receive a sample. The sample can be a lower nasal swab sample, nasopharyngeal swab sample, gingival swab sample, buccal swab sample, gargle sample, sputum sample, or saliva sample, for example, though other sample types may be utilized according to application preferences and specifics. The buffer tube 108 includes a sample processing buffer formulated to prepare the sample for the subsequent amplification reactions within the reaction card.

The outer cover 102 can include an openable/closable lid 110. The reaction chambers 114 and a readout card 112 are visible underneath the lid 110. An example readout card 112 is shown in FIG. 1B from a top-down view. The example readout card 112 overlays (and/or is adjacent to) multiple reaction chambers 114 in which nucleic acid amplification and detection reactions occur. Reaction chamber labels 116 indicate the target (e.g., pathogen) tested in the corresponding reaction chamber 114. The device 100 can include multiple reaction chambers 114 and can simultaneously test for multiple targets. As shown, the device 100 can include a positive control reaction chamber and multiple target reaction chambers. Example targets include SARS-CoV-2, Flu (e.g., A and/or B), and Respiratory Syncytial Virus (RSV), though additional or alternative targets can be included depending on particular application needs.

The readout card 112 includes a colorimetric results label 118 for illustrating the expected reaction chamber coloration for a negative result and positive result. The particular colors of the test will depend on the readout indicator formulation included in the reaction chambers 114. Examples of readout indicator formulations are described below and include pH-dependent and pH-independent chemistries. While any suitable readout protocol can be utilized (e.g., fluorescence-based methods), presently preferred embodiments include colorimetric readout indicators that can be directly visualized by the naked eye.

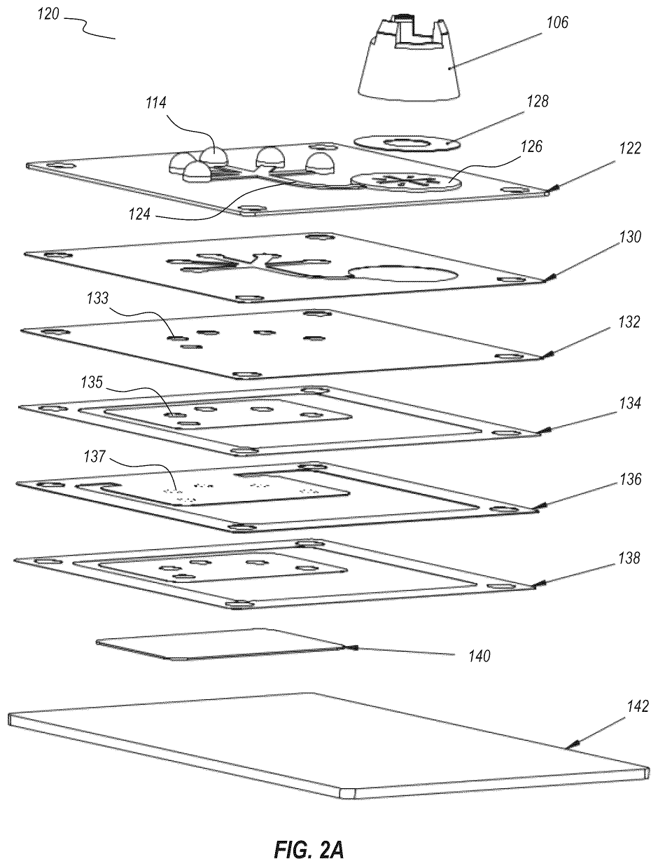

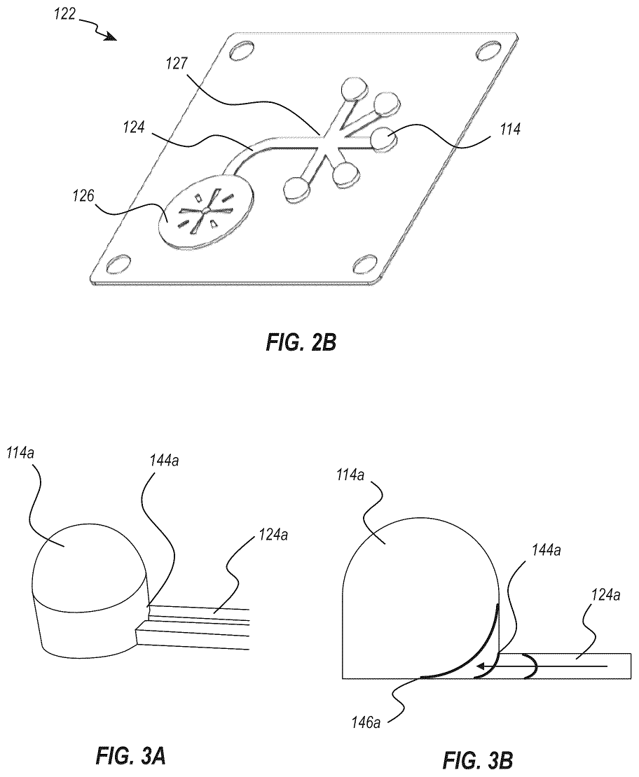

FIG. 2A is an exploded view of a reaction card 120 configured to receive a sample mixture from the buffer tube 108 (e.g., via receiving base 106) and direct the sample mixture to the reaction chambers 114 where amplification and detection reactions can occur. The reaction card 120 can be designed with a laminate structure (i.e., comprising multiple layers). In the illustrated example, a card top 122 is structured (e.g., with grooves and/or raised portions) defining microfluidic channels 124 that extend between the receiving area 126 and the reaction chambers 114. The receiving base 106 can be coupled to the receiving area 126 with an adhesive layer 128, as shown. Alternatively, the receiving base 106 can be integrally formed as part of the card top 122.

In the illustrated embodiment, a film layer designed as a channel backing 132 is disposed below the card top 122. The channel backing 132 defines the bottom of the microfluidic channels 124. A channel adhesive layer 130 can be disposed between the card top 122 and the channel backing 132 and used to connect these layers. As shown, the channel adhesive layer 130 includes cutouts to account for the microfluidic channels 124 and the receiving area 126 so that these areas remain open between the card top 122 and the channel backing 132. The channel backing 132 includes apertures 133 corresponding to the overlying reaction chambers 114. The apertures 133 allow for venting of gasses and fluid to lower layers of the device.

A film layer designed as a chamber bottom 136 is disposed beneath the channel backing 132. A chamber adhesive layer 134 can be disposed between the channel backing 132 and the chamber bottom 136 and used to connect these layers. The chamber adhesive layer 134 includes apertures 135 aligned with the apertures 133 of the channel backing 132. The chamber bottom 136 includes a surface that defines the bottom of the reaction chambers 114. In some embodiments, the relative positions of the channel backing 132 and the chamber bottom 136 can be reversed, or alternatively, these layers can be combined as a single layer that defines the bottom of the microfluidic channels 124 and the reaction chambers 114.

The chamber bottom 136 includes vent holes 137, which may be in the form of slits, pinholes, and/or other openings, to allow passage of air from the reaction chambers 114 into the venting membrane 140. The venting membrane 140 is attachable to the chamber bottom 136 with a vent adhesive layer 138. The vent adhesive layer 138 includes apertures 139 that align with the vent holes 137. The venting membrane 140 is configured to allow escape of gasses for pressure equalization during fluid flow while maintaining a barrier against liquids, dust, or other contaminants. The venting membrane 140 can be a polytetrafluoroethylene (PTFE) membrane, for example, or can additionally or alternatively include other materials such as polyethylene (PE), polypropylene (PP), polyethylene terephthalate (PET), polyurethane (PU), ceramics, or other suitable venting membrane materials.

A card bottom 142 attaches to the chamber bottom 136 (e.g., via the outer portions of the vent adhesive layer 138) and defines the bottom of the reaction card 120. In some embodiments, the card bottom 142 can incorporate a heat source, such as a printed circuit board (PCB) with heat element traces. More preferred embodiments, such as described below, utilize a chemical-based heat source, such as a heat source based on an exothermic reaction.

The card top 122, card bottom 142, and/or other rigid portions of the reaction card 120 can be formed from any suitable material or set of materials that are amenable to forming laminate structures, including polymers such as polycarbonate (PC) or other suitable structural polymer materials. Film layers, such as the channel backing 132 and chamber bottom 136 can be formed from any suitable film material, such as polyester film and/or other suitable polymer materials. A “rigid” material refers to a material that withstands deformation under normal manual manipulation of the reaction card as described herein. A “film” refers to a material that is more flexible that a rigid material and can readily be deformed under normal manual manipulation.

FIG. 2B is another view of the top card 122 to better illustrate the reaction chambers 114, microfluidic channels 124, and receiving area 126. The illustrated embodiment shows five separate reaction chambers 114. Other embodiments can include fewer or more reaction chambers 114, depending on desired assay targets and/or particular application requirements. The illustrated embodiment includes a microfluidic channel 124 that follows a common channel before splitting in a radial fashion into multiple sub-channels, each corresponding to a respective reaction chamber 114, at a junction point 127. While this design has proven to be effective, other channel designs are also usable, such as designs that include multiple junction points and/or that distribute sub-channels in linear (as opposed to radial) fashion.

During use of the device 100, insertion of the buffer tube 108 into the receiving base 106 causes release of the sample mixture (the sample processing buffer mixed with the sample) into the microfluidic channels 124. Capillary forces act on the sample mixture to pull it through the microfluidic channels 124 and into the reaction chambers 114.

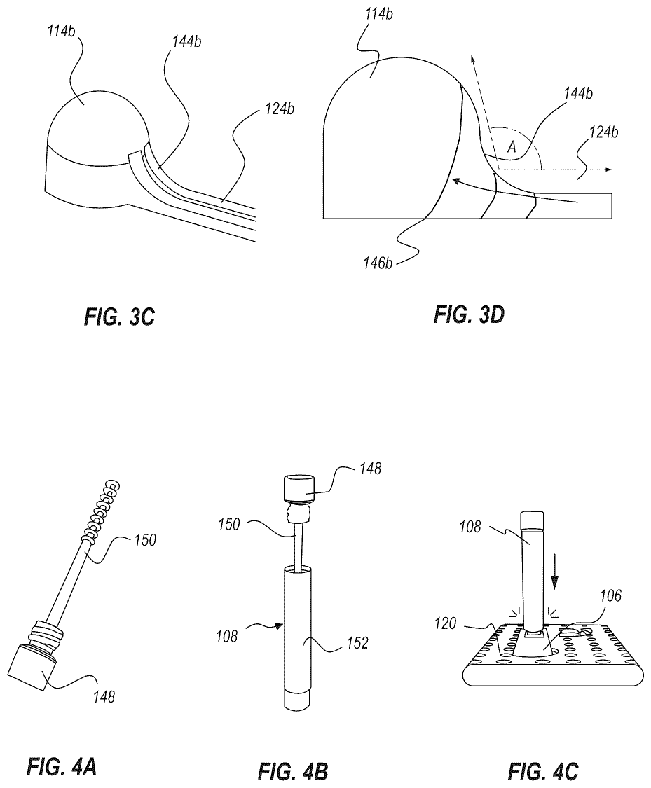

FIGS. 3A through 3D illustrate more detailed views of different example reaction chambers 114a and 114b. In FIG. 3A, the joint 144a where microfluidic channel 124a connects to reaction chamber 114a forms a “sharp” corner of approximately 90°. As illustrated in FIG. 3B, the advancing wetting front 146a (different lines showing advancement over time) tends to get pinned at the sharp cornered joint 144a until the bottom portion of the wetting front 146a advances far enough to bring the rest of the front into the reaction chamber 114a.

In contrast, the configuration shown in FIG. 3C includes a fillet structure at joint 144b. The fillet structure imparts a gradual curve to the joint 144b and avoids the sharp corner of joint 144a. As shown in FIG. 3D, the advancing wetting front 146b (different lines showing advancement over time) is better able to enter the reaction chamber 114b with reduced pinning at the joint 144b. This beneficially allows for more efficient wetting of the reaction chamber 114b, better contact of sample mixture to the master mix within the reaction chamber 114b, and better consistency and reproducibility of results across different reaction cards and samples.

Whereas the angle at joint 144a is approximately 90°, the angle (A) imparted by the fillet structure at joint 144b can be greater than 90°, such as about 110° to about 155°, or about 115° to about 145°, or about 120° to about 135°, or within a range using any combination of the foregoing values as endpoints.

The design of the assay device 100 beneficially allows testing to be carried out without requiring manual transfer steps such as pipetting. The passive microfluidics work in tandem with the specific shape and volume of the reaction chambers 114 to effectively control fluid/reagent movement. This enables effective performance of the reactions carried out in the reaction chambers 114 during use of the assay device 100.

In addition, the assay device 100 omits hinges, multiple seals, and other extraneous components that complicate the ability to quickly carry out the intended reaction. For example, the assay device 100 does not require the manual removal of reaction chamber seals, the opening/closing of various reaction chamber caps, or the manual pipetting of liquids into the reaction chambers 114. This beneficially saves user time and reduces the risk of workflow error.

### Assay Device Operation

FIGS. 4A through 4C illustrate an example workflow for initiating a test with the assay device 100. The buffer tube 108 can include a cap portion 148 and an internal swab portion 150 connected to and extending from the cap portion 148. The cap portion 148 can include threads and/or other fixture elements (e.g., snap-fit structures, adhesive, magnetic couplings) that enable connection to the body portion 152 of the buffer tube 108.

After disconnecting the cap portion 148 and swab portion 150 from the body portion 152, a test subject or caretaker can obtain a swab sample (e.g., a nasal swab sample) using standard swab protocols known in the art. For example, the swab portion 150 can be inserted a sufficient distance into the nasal passages of the subject and rotated to gather sufficient sample, as illustrated in FIG. 4A. The swab portion 150 can then be placed within the body portion 152 and the cap portion 148 can be locked in the closed position, as shown in FIG. 4B.

Although this example is configured for receiving a nasal swab, it will be understood that other embodiments can additionally or alternatively utilize other sample types. In some embodiments, the sample can be a lower nasal swab sample, nasopharyngeal swab sample, gingival swab sample, buccal swab sample, gargle sample, sputum sample, or saliva sample. In some embodiments, the sample may include other bodily fluids depending on the types of diseases, biomarkers, or pathogens specific to the assay. In some embodiments, the sample can be an environmental sample collected from soil or water, for example. In some embodiments, the sample can be agricultural or food products such as feedstocks, vegetables, fruits, meat, milk, honey, etc. In some embodiments, the target of the molecular diagnostic test may be viruses, bacteria, algae, or other types of microorganisms.

The body portion 152 contains a sample processing buffer. When the sample is mixed with the sample processing buffer, the resulting sample mixture can be utilized in downstream amplification and detection reactions. The sample processing buffer can be formulated to effectively inactivate and lyse antimicrobial elements of the sample (e.g., viral particles) and stabilize the released nucleic acid material under proper conditions (including temperature and pH) that are also compatible for a simultaneous nucleic acid amplification reaction to take place without intervening purification steps and without inhibition or cross-reactivity.

As shown in FIG. 4C, the buffer tube 108 can then be attached to the reaction card 120 to initiate movement of the sample mixture through the microfluidic channels 124 to the reaction chambers 114. The body portion 152 of the buffer tube 108 is configured in size and shape to engage with the receiving base 106. This can be accomplished through a snap-fit engagement, threaded connection, and/or other suitable means of fixation. In some embodiments, the buffer tube 108 includes a frangible seal that is punctured during insertion into the receiving base 106 to allow the sample mixture to pass into the reaction card 120.

Capillary action provided by the design of the reaction card 120 moves the sample mixture into the reaction chambers 114, which can be pre-loaded with a master mix to enable nucleic acid amplification to occur within the reaction chambers 114.

Addition of the sample mixture to the master mix can initiate a one-pot reaction that allows both the sample processing (including sample inactivation, sample lysis, nuclease inhibition, nucleic acid extraction, nucleic acid stabilization) and the nucleic acid amplification (e.g., LAMP, RT-LAMP) to take place within the same reaction chamber by incubation at a single temperature (e.g., 60-68° C.) for a short period of time (e.g., 15-45 minutes).

Subsequently, the result of the test can be read by direct naked eye (colorimetric) or device-assisted (e.g., colorimetric, fluorescent, or electrochemical) using an analysis device such as a handheld computer device interpretation. In some embodiments, the result may be qualitative (yes-or-no) based on a binary readout, whereas in some other embodiments, the readout result may be semi-quantitative or quantitative. Presently preferred embodiments provide for direct naked-eye qualitative determinations (see, e.g., FIG. 1B) based on pH-dependent or pH-independent colorimetric readout chemistries.

The assay device 100 can therefore provide simple and easy operation that (i) processes the sample, (ii) releases and amplifies target nucleic acids, and (iii) activates a readout indicator. When used in conjunction with the electricity free heating/insulation features described elsewhere herein, the assay device 100 can provide simple, easy, and economical assay testing with rapid results, without the need for sophisticated and expensive laboratory equipment, and even without the need for electricity or batteries.

The assay device 100 can be configured to carry out an isothermal nucleic acid amplification reaction. For example, the assay device 100 can be configured to carry out one or more loop-mediated isothermal amplification (LAMP) and/or reverse-transcription LAMP (RT-LAMP) reactions. Other amplification methods may additionally or alternatively be carried out, such as dual-priming isothermal amplification (DAMP), cross-priming amplification (CPA), strand displacement amplification (SDA), rolling circle amplification (RCA), recombinase polymerase amplification (RPA), helicase-dependent amplification (HDA), nucleic acid sequence-based amplification (NASBA), multiple displacement amplification (MDA), whole genome amplification (WGA), genome exponential amplification reaction (GEAR), exponential amplification reaction (EXPAR), nicking and extension amplification reaction (NEAR), single chimeric primer isothermal amplification (SPIA), isothermal and chimeric primer-initiated amplification of nucleic acid (ICAN), hairpin fluorescence probe-assisted isothermal amplification (PHAMP), signal-mediated amplification of RNA technology (SMART), beacon-assisted molecular detection (BAD AMP), CRISPR-Cas9-triggered nicking endonuclease-mediated strand displacement amplification (CRISDA), as well as enzyme-free nucleic acid amplification methods such as hybridization chain reaction (HCR), catalyzed hairpin assembly (CHA), exponential hairpin assembly (EHA), entropy-driven catalysis (EDC) such as toehold-mediated strand displacement (TMSD), and combinations thereof.

Example LAMP and RT-LAMP methods that can be utilized by the disclosed assay device are described in U.S. patent application Ser. No. 17/749,858, titled “Universal Lamp Assays for Detection of Nucleic Acid Targets,” which is incorporated herein by reference in its entirety. An example method based on an EXPAR approach is described in U.S. Provisional Application No. 63/550,424, titled “Ultra-Fast One-Pot Exponential Isothermal Amplification of Nucleic Acids,” and which is incorporated herein by reference in its entirety.

### Heating/Insulation

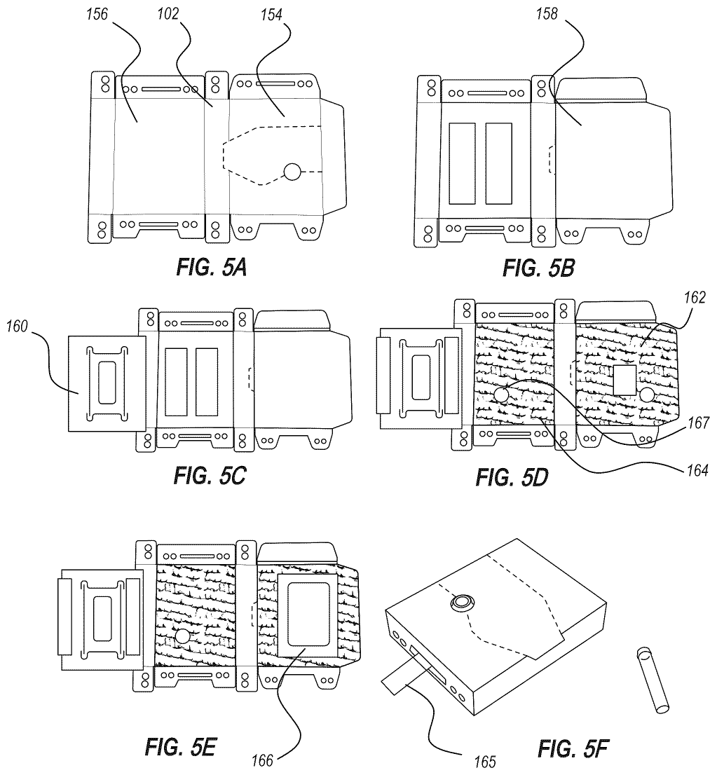

FIGS. 5A through 5F illustrate a process for assembling the outer cover 102 and internal components of the assay device 100. The outer cover 102 can be formed from paper or cardboard that has sufficient weight and/or edge crush test (ECT) rating to enable expected stacking, transport, and field use of the assay device 100.

In the illustrated example, a cutout of the outer cover 102 is laid out (FIG. 5A) with inner surfaces of a top panel 154 and bottom panel 156 shown. Adhesives 158 (e.g., double sided) are added (FIG. 5B). An interior stiffener 160 can be attached to the bottom panel 156 (FIG. 5C). The reaction card 120 (not visible in this view) is then positioned on the top panel 154 with receiving base 106 protruding through the aperture 104 (see FIG. 1A). An upper insulation layer 162 and lower insulation layer 164 are added to respective top panel 154 and bottom panel 156 (FIG. 5D). One or more spacers 167 can also be added to provide defined space between the upper and lower insulation layers 162, 164. A temperature regulator (not visible) and heat source 166 are then attached (FIG. 5E), with the temperature regulator positioned between the heat source 166 and the reaction card 120. The outer cover 102 is then folded to form the finished assay device 100 (FIG. 5F).

As shown in FIG. 5F, a pull tab 165 can be routed through the outer cover 102 and connected to the heat source 166. The heat source 166 can be easily activated by pulling the pull tab 165 to allow air to contact the heat source 166 and initiate the exothermic reaction.

The interior stiffener 160 can be folded to be positioned between the lower insulation layer 164 and the heat source 166. The interior stiffener 160 can beneficially assist in allowing sufficient upward/downward movement of the temperature regulator and/or heat source 166 during operation. Such movement and its benefits are described in more detail below.

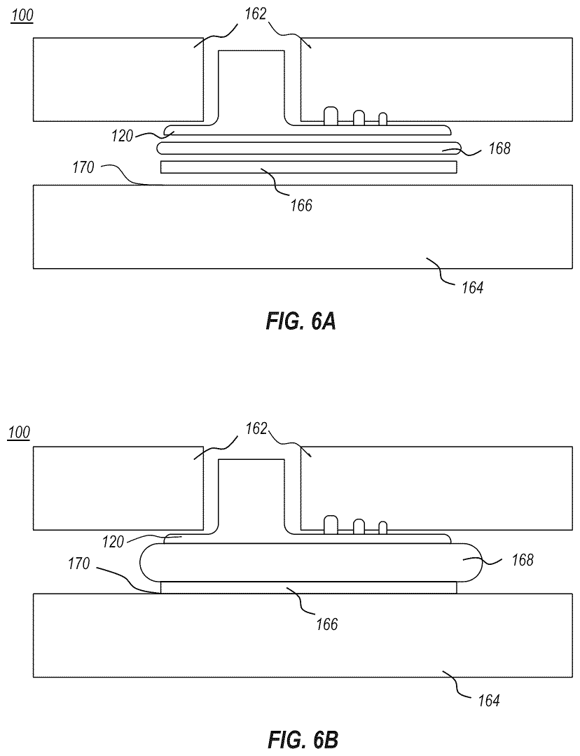

FIG. 6A illustrates a cross-sectional side view of the assay device 100, showing the temperature regulator in the form of a pouch 168 connected to the heat source 166. The heat source 166 is preferably an electricity free heat source, such as a pouch or other container that selectively carries out an exothermic reaction upon sufficient exposure to oxygen. The pouch 168 includes an inner cavity that houses a solid carrier and a phase change material. A first (e.g., bottom) side of the pouch 168 is in thermal contact with the heat source 166, and a second (e.g., upper) side of the pouch 168 is in thermal contact with the reaction card 120. The heat source 166 can be activated to supply heat to the pouch 168, causing the phase change material to undergo a phase change from liquid to gas, thereby causing the pouch 168 to expand. An air inlet 170 is provided by allowing space between the lower insulation layer 164 and the heat source 166. This space can be provided, for example, by one or more spacers 167 (see FIG. 5D) between the upper and lower insulation layers 162, 164.

Because the phase change material within the pouch 168 is tuned to evaporate at or slightly above the target reaction temperature, the principles of evaporation promote a substantially constant temperature transferred the reaction card 120. That is, while the phase change material undergoes a phase change from liquid to gas, the resulting temperature will be approximately equal to the boiling point of the phase change material. Thus, by tuning the phase change material to have a boiling point at the targeted reaction temperature (or slightly above to account for slight heat transfer losses), the temperature regulator can buffer any excesses in temperature output by the heat source 166 and maintain a substantially constant reaction temperature at the reaction card 120.

FIG. 6B shows the same view of the assay device 100 after the heat source 166 has been activated and after the pouch 168 has expanded from being heated. The expanded pouch 168 causes the heat source 166 to move toward an interior surface of the housing 101, reducing the size of the air inlet 170, thereby restricting the flow of oxygen powering the heat source 166. This beneficially prevents the heat source 166 from expending too much heat in too short a time, and instead allows the heat source 166 and pouch 168 of the temperature regulator to maintain the target temperature for a more sustained duration. At the same time, if the heat source 166 begins to cool by too much, the pouch 168 will begin to shrink before any significant change in its phase change regulated temperature. Such shrinking will expand the size of air inlet 170 and allow the heat source 166 to generate more heat.

To promote sufficiently high temperatures, the heat source 166 is preferably configured to deliver a temperature that is higher than the targeted reaction temperature at the reaction card 120, while allowing the temperature regulator to buffer the excess heat. Despite the preference for running the heat source 166 at a temperature that is higher than the reaction temperature, it is still desirable to limit temperatures that are too high with respect to the desired temperature difference and/or that cause overly rapid depletion of the exothermic reaction. The illustrated configuration thus provides thermal regulation via evaporative principles as well as self-regulation of the amount of oxygen driving the exothermic reaction.

The assay device 100 therefore allows the heat source 166 to heat up relatively quickly while the pouch 168 is not in an expanded configuration. As the pouch 168 starts to expand as it is heated, the air inlet 170 becomes progressively more restricted, regulating the power output of the heat source 166, thereby conserving the heat source 166 and allowing for a longer lasting delivery of heat. This better maintains desired isothermal conditions, for a longer duration, to carry out isothermal reactions at the reaction card 120.

In the illustrated embodiment, the air inlet 170 closes completely upon maximum expansion of the pouch 168. Alternatively, the size of the pouch 168 and the air inlet 170 can be tailored so that a gap remains between the heat source 166 and the lower insulation layer 164 even at maximum expansion of the pouch 168. For example, the air inlet 170 may gradually diminish as the pouch 168 expands, and therefore progressively lower the amount of oxygen available for the heat source 166, while still avoiding completely closing off the air inlet 170.

The pouch 168 may include a polyester-based film such as a polyethylene terephthalate (e.g., biaxially oriented polyethylene terephthalate (BoPET), known by the trade name MYLAR), polypropylene, nylon, polyethylene, and/or polypropylene, and may additionally or alternatively include other materials known in the art as suitable for film applications. For example, the film may be additionally or alternatively made of other polyester materials and/or other polymer materials appropriate as films for a temperature regulation device. The film may be metalized. A metalized film includes a thin coating of metal, typically aluminum, though other metals such as nickel and/or chromium may be utilized.

The solid carrier disposed inside the inner cavity of the pouch 168 can be formed of a suitable fabric, such as a poly broadcloth, a polyester/cotton blend fabric, and/or other fabric material capable of holding a solvent phase change material. In some embodiments, the solid carrier may additionally or alternatively include (e.g., non-fabric) solid materials such as, for example, absorbent beads, granular materials, sponge materials, paper or other cellulose material, fibrous materials, fiber bundles, or combinations thereof. Other embodiments may omit a solid carrier and simply include a phase change material directly added to the inner cavity of the pouch 168. The solid carrier can be soaked or dampened with the phase change material.

The phase change material may include, for example, an inorganic solvent and/or an organic solvent. Suitable solvents include methanol, n-hexane, cyclohexane, ethanol, ethyl acetate, isopropanol, tert-butanol, benzene, tetrahydrofuran, other solvents, or combinations thereof. Generally, solvents are suitable where they exhibit relatively minimal toxicity and have boiling points at or slightly higher than the target temperature of the intended reaction. Solvent mixtures may also be utilized, particularly where the mixed solvents have similar boiling points. With solvent mixtures, one or more solvents of the mixture may exhibit a boiling point outside the target range of the intended reaction, but the overall temperature regulation effect of the mixture can still be effective for maintaining the reaction within the target temperature range.

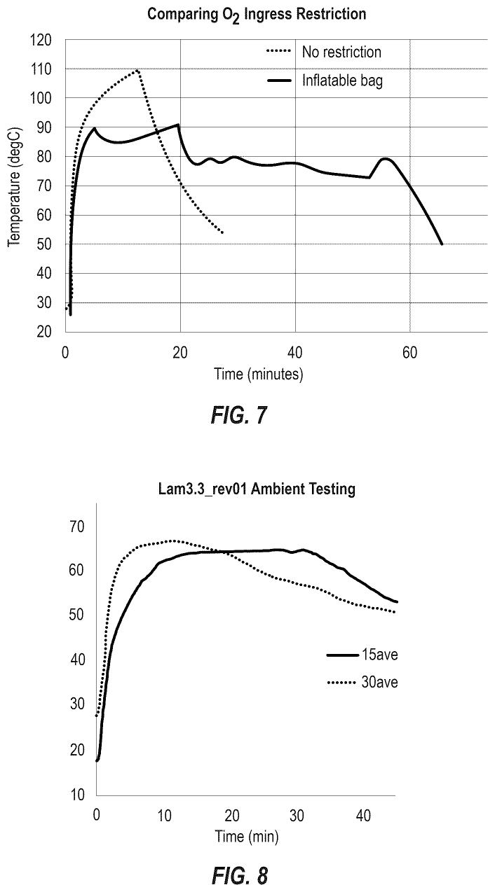

For example, LAMP or RT-LAMP reactions are carried out at a temperature between about 60° C. and about 70° C. The assay device 100 can maintain a temperature at the reaction card 120 in this range for over an hour, which is more than enough to carry out a full LAMP or RT-LAMP reaction. This is illustrated by FIG. 7, which compares the temperature over time profile of a heat source with unrestricted access to air, and a heat source with a restricted oxygen inlet. The restricted heat source provides more stable and longer-lasting heat delivery.

The solvent or solvent mixture can therefore be tailored to arrive at a desired boiling point. As an example for LAMP or RT-LAMP applications, methanol has a boiling point of about 65 degrees Celsius. Some embodiments can additionally or alternatively include ethanol and/or isopropanol as the phase change material. Ethanol and isopropanol have higher boiling points (about 78 degrees Celsius and about 82 degrees Celsius, respectively), but can still be utilized in at least some applications for effective temperature regulation within the target LAMP/RT-LAMP range of 60 to 70 degrees Celsius.

During use, the temperature of the reaction will tend to be slightly lower than the temperature of the pouch 168 due to inherent heat transfer losses. Thus, in some implementations, the phase change material may be formulated with a boiling point that is slightly above the target temperature of the intended reaction. For example, the phase change material can be formulated with a boiling point that is 0.1, 0.5, 1, 2.5, 5, 7.5, 10, 12.5, or 15 degrees Celsius (or a range using any combination of the foregoing as endpoints) higher than a target reaction temperature. This intentional offset can account for such heat transfer effects and bring the reaction temperature closer to the intended target temperature.

FIG. 8 illustrates temperature profiles for assay devices tested at 15° C. ambient environment and 30° C. ambient environment. FIG. 8 shows the average of n=3 for each temperature condition. The results show a sustained and substantially stable temperature over time periods conducive to isothermal reaction applications.

Additional details regarding temperature regulation devices and methods for heating the assay device are provided in U.S. patent application Ser. No. 18/205,992, titled “Biochemical Reaction Temperature Regulator with Liquid-Gas Phase Change Material,” and U.S. Provisional Patent Application No. 63/606,214, titled “Biochemical Reaction Temperature Regulator with Stabilized, Self-Regulating Heat Source,” each of which is incorporated herein by reference in its entirety.

### Example Reagent Details

The sample processing buffer can comprise any combination of: a surfactant comprising Tween 20, Tween 80, Triton X-100, Triton X-114, NP-40, Igepal CA-630, CHAPS, and/or SDS; a reducing/denaturing agent comprising DTT, TCEP, urea, GuHCl, GITC, and/or formamide; optionally, a nuclease inhibitor comprising proteinase K, murine RNase inhibitor, human placenta RNase inhibitor, VSA, PVSA, ACP, RNasin® Plus Ribonuclease Inhibitor, RiboGrip RNase Inhibitor, RiboLock RNase Inhibitor, SUPERase·In, RNaseOUT, and/or RNAsecure; a chelating agent such as EDTA; a buffering salt comprising Tris, Tris-HCl, TE, TAE, TBE, and/or a solution of HCl, NaOH, and/or KOH; and a molecular enhancer such as polyethylene glycol (e.g., PEG 8000).

The master mix can comprise any combination of: MgSO₄ or MgCl₂; (NH4)₂SO₄ or (NH4)₂Cl₂; dNTP mix, optionally included at 1 mM to 2 mM; DNA polymerase, the DNA polymerase optionally comprising Bst 2.0 or Bst 2.0 WarmStart DNA Polymerase; Tween 20 or Triton-100; Tris-HCl, or Tris or TE; KCl; betaine; primers for specific amplification of a target nucleic acid sequence from the sample; reverse transcriptase; GuHCl; Antarctic Thermolabile Uracil-DNA-glycosylase (UDG) and dUTP; one or more reaction enhancers comprising crowding agents, dsDNA destabilizers, dsDNA stabilizers, enzyme stabilizers, template blockers, and/or oligonucleotide modifications or analogs; and one or more excipients comprising sucrose, trehalose, dextran, pullulan, lactose, glucose, raffinose, mannitol, sorbitol, glycine, histidine, arginine, gelatin, dextrose, hydroxyethyl starch, poly(ethylene glycol), poly(propylene glycol), and/or poly(vinyl alcohol).

In some embodiments, the master mix is formulated as a LAMP or RT-LAMP master mix comprising any combination of: MgSO₄ (or MgCl₂), (NH4)₂SO₄ (or (NH4)₂Cl₂), dNTP mix, dUTP, thermolabile UDG, DNA polymerase (e.g., Bst 2.0, Bst 3.0, or Bst 2.0 WarmStart® DNA Polymerase), Tween 20 (or Triton-100), Betaine, Tris-HCl, and KCl.

In some embodiments, the master mix comprises a commercially available LAMP or RT-LAMP master mix including but not limited to WarmStart® Multi-Purpose LAMP/RT-LAMP 2× Master Mix with UDG (NEB M1708), WarmStart® Colorimetric LAMP 2× Master Mix with UDG (NEB M1804), Invitrogen™ SuperScript™ IV RT-LAMP Master Mix (A51802), and their lyo-ready, glycerol-free, and high-concentration versions. In some embodiments, the master mix comprises a reverse transcriptase (e.g., NEB WarmStart® RTx Reverse Transcriptase) to form an RT-LAMP master mix for detection of RNA.

In some embodiments, the master mix comprises an additional high-performance strand-displacing DNA polymerase such as Invitrogen™ Lyo-ready Bst DNA Polymerase (A56656) or its equivalent versions (e.g., standard, lyo-ready, glycerol-free, and high-concentration) and/or an additional high-performance reverse-transcriptase such as Invitrogen™ Lyo-ready SuperScript™ IV Reverse Transcriptase (EP164B1B008) or its equivalent versions (e.g., standard, lyo-ready, glycerol-free, and high-concentration).

In some embodiments, the master mix comprises a set of LAMP/RT-LAMP primers including forward outer primer (i.e., F3 primer), backward outer primer (i.e., B3 primer), forward inner primer (i.e., FIP primer), and backward inner primer (i.e., BIP primer) formulated for specific amplification of a target nucleic acid sequence from the sample, and optionally further comprises one or more loop primers (i.e., LoopF primer and/or LoopB primer). The master mix can further include one or more accelerating primers, such as in the form of stem primers, swarm primers, and/or alternative primers that function based on similar mechanisms that facilitate acceleration of the LAMP/RT-LAMP reaction.

Primers included in the master mix can be provided as sets of primers targeting different regions of a specific target nucleic acid sequence or targeting different sequences from different target samples, in a multiplexed fashion.

The readout indicator can comprise any combination of: a pH indicator, optionally selected from Phenol Red, Neutral Red, Cresol Red, Cresol Purple, Thymol Blue, Bromothymol Blue, Bromophenol Blue, Litmus, Chlorophenol Red, Dichlorofluorescein, Methyl Red, Bromocresol Purple, Naphtholphthalein, and/or Cresolphthalein; a metal indicator that senses metal ions such as Mg²⁺, Mn²⁺, Zn²⁺, Cu²⁺, Co²⁺, Cd²⁺, Fe²⁺, Ni²⁺, Hg²⁺, Pb²⁺, such as a composition comprising one or more of (i) Hydroxynaphthol Blue, (ii) Eriochrome Black T, (iii) Calcein, or (iv) pyridylazophenol dye such as 2-(5-Bromo-2-pyridylazo)-5-[N-propyl-N-(3-sulfopropyl) amino]phenol (5-Bromo-PAPS) or 2-(5-Nitro-2-pyridylazo)-5-[N-n-propyl-N-(3-sulfopropyl)amino]phenol (5-Nitro-PAPS); a fluorescent/colorimetric DNA binding dye such as SYBR Gold, SYBR Safe, Leuco Crystal Violet, Malachite Green, Methyl Green, EvaGreen, SYTO 9; and/or a nanoparticle-based indicator such as gold nanoparticles.

Additional details regarding sample processing buffer, master mix, and readout indicator chemistries are provided in U.S. patent application Ser. No. 18/370,117, titled “Rapid Molecular Diagnostics with Unified-One-Pot Sample Processing, Nucleic Acid Amplification, and Result Readout,” and in U.S. Provisional Patent Application No. 63/618,116, titled “Ultra-Fast One-Pot Loop-Mediated Isothermal Amplification and Detection of DNA and RNA,” each of which is incorporated herein by reference in its entirety.

The master mix can be prepared in wet, frozen, air dried, or lyophilized form. In presently preferred embodiments, the master mix is lyophilized in place within the respective reaction chambers 114. By lyophilizing the reagent in place, the dry reagent can be positioned very close (e.g., as close as possible) to the point where the wetting front enters the corresponding reaction chamber 114. Keeping this distance minimized and controlled enables effective conditions for wetting the lyophilized master mix. In contrast, with spherical beads and corresponding pick-and-place assembly, as used in conventional assay designs, the inherent clearance between the lyophilized spherical pellets and the walls of the reaction chamber can introduce delays and variability the time it takes the wetting front to traverse the space between the entrance of the reaction chamber and the outer surface of the lyophilized bead. These increased variability and relative delays negatively affect the overall function of the associated assay device.

### In-Device Lyophilization

An example method for preparing an assay device that includes lyophilized reagents comprises dispensing a reagent mixture into a reaction chamber, the reaction chamber having a specified volume and shape, and subjecting the reaction chamber to temperature and pressure conditions sufficient to cause lyophilization of the reagent mixture and formation of a lyophilized reagent pellet in place within the reaction chamber, wherein the resulting lyophilized reagent pellet matches the shape of the reaction chamber.

The assay device can include a single reaction chamber or multiple reaction chambers. For example, an assay device comprising multiple reaction chambers can include different reaction chambers each with distinctly formulated reagent mixtures for assaying different pathogens, providing various controls, and/or providing alternative readout types. For assay devices that include multiple reaction chambers, the reagent mixtures of at least one of the reaction chambers, or preferably of all the reaction chambers, are lyophilized in place according to the disclosed method.

Subjecting the reaction chamber to temperature and pressure conditions sufficient to cause lyophilization can include contacting a surface of the assay device and/or reaction card, directly or indirectly, with a suitable coolant composition (e.g., liquid nitrogen, dry ice, and/or other suitable coolant composition). For example, following placement of the one or more reagent mixtures in their respective reaction chambers, an outer surface of the reaction card can be directly contacted with the coolant composition and/or an outer surface of the reaction card can be contacted with a cooled surface (e.g., a surface that is itself cooled by a suitable coolant composition, Peltier element, and/or some other refrigeration means).

In some embodiments, the coolant composition is contacted to a bottom surface of the reaction card while the reaction chamber(s) on the top surface of the assay device remain open. This can avoid contact between the coolant composition and the reagent mixture(s). In some embodiments, the coolant composition can be added directly to the reagent mixtures. The reaction chambers with frozen reagent mixtures can then be subjected to temperature and pressure conditions sufficient to complete the lyophilization.

Beneficially, each resulting lyophilized pellet will match the shape of the reaction chamber in which it is disposed. This contrasts with the conventional process of preparing dry storage reagents for assay devices, where lyophilized beads must be separately manufactured, stored, transported, and loaded into the reaction chambers, and the tolerances of the reaction chambers and beads must be controlled to ensure sufficient clearance for fitting the beads inside the reaction chambers.

The disclosed embodiments also provide improvements relative to non-bead shaped lyophilized materials. For example, the product sold under the trade name LyoDoT (Argonaut Manufacturing Services, Carlsbad, CA) includes flat, circular-shaped lyophilized “dots” generated by applying liquid reagent to a treated film surface and then lyophilizing the reagent. However, the dots must be manufactured separate from the assay device followed by removal and placement/integration into the assay device. Moreover, while the flat, circular shape of the dots can be beneficial relative to conventional beads, the dots are unlikely to match the shape of the reaction chambers to which they are added.

In contrast, the presently described embodiments enable manufacture of the lyophilized materials directly within the reaction chambers, eliminating the need to first form the lyophilized materials externally and subsequently integrate them into the reaction chambers. The lyophilized materials of the present disclosure also form shapes that match their respective reaction chambers. This eliminates the need to design reaction chambers and/or other components of the assay to meet requirements based on pre-defined size and shape of the lyophilized materials. Rather, reaction chamber dimensions can be readily customized according to other pertinent assay design requirements.

One example feature that can be optimized using the disclosed embodiments is the liquid pathway for rehydrating the lyophilized reagent. By lyophilizing the reagent in place, the dry reagent can be positioned very close (e.g., as close as possible) to the point where the wetting liquid front enters the reaction chamber. Keeping this distance minimized and controlled enables effective conditions for wetting the lyophilized reagents. In contrast, with spherical beads and corresponding pick-and-place assembly, the inherent clearance between the lyophilized spherical pellets and the walls of the reaction chambers can introduce delays and variability the time it takes the wetting liquid front to traverse the space between the entrance of the reaction chamber and the outer surface of the lyophilized bead. These increased variability and relative delays negatively affect the overall function of the associated assay device.

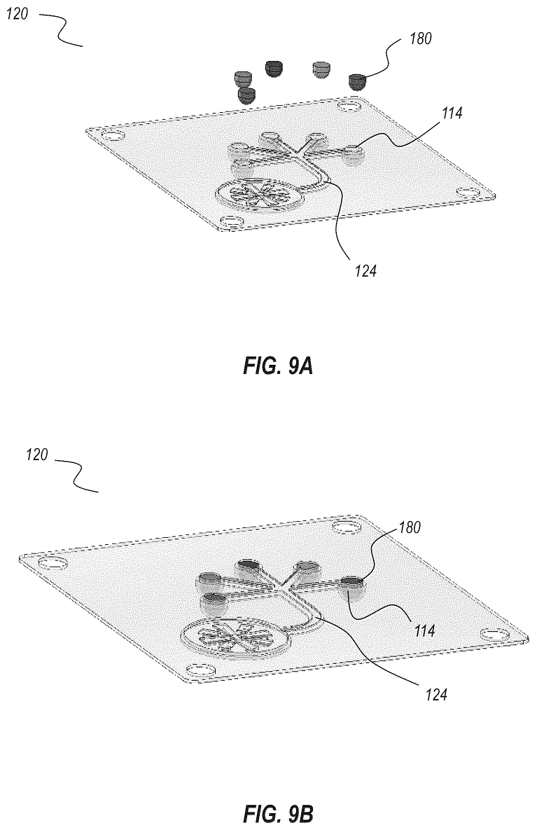

FIGS. 9A and 9B illustrate the example reaction card 120 after an in-device lyophilization process, with FIG. 9A showing an exploded view and FIG. 9B showing an intact view. As shown, the lyophilized reagent pellets 180 match the shape of their corresponding reaction chambers 114.

In this example, the reaction chambers 114 are formed as recesses within a bottom portion 106 of the reaction card 120. After dispensing of the reagent mixtures into the reaction chambers 114 and carrying out lyophilization to form the lyophilized reagent pellets 180, an upper portion (not shown) can be attached to the bottom portion to form the full card.

Forming the lyophilized reagent pellet within the reaction chambers 114 beneficially eliminates the intermediate manufacturing, storage, and transport steps between lyophilized bead production and final bead loading into the assay device. This results in a faster, less costly, and less complicated manufacturing process. In particular, the disclosed in-device lyophilization process does not require a solid bead to be inserted into the device during production.

In addition to the simplification of the manufacturing workflow, the void-filling nature of the modified geometry of the lyophilized reagent pellet can provide more control over the drying process and improved rehydration performance when the assay device is used. For example, because the lyophilized reagent mixture takes the shape of its reaction chamber container, the reaction chamber can be optimized to improve other aspects of the assay device such as visual readout, wetting, rehydration volume, heat transfer, overall device footprint, and/or other aspects of the assay.

### EXAMPLE EMBODIMENTS

Clause 1. An assay device configured for isothermal amplification and detection of nucleic acids, the assay device comprising: a buffer tube containing a sample processing buffer, the sample processing buffer formulated for mixing with a sample; and a reaction card configured to receive the buffer tube and at least a portion of its contents, the reaction card comprising a receiving area for receiving the contents of the buffer tube, one or more reaction chambers each containing a master mix formulated to enable amplification of a target nucleic acid, and one or more channels fluidically connecting the receiving area with the one or more reaction chambers and configured to deliver the received contents of the buffer tube to the one or more reaction chambers, wherein the one or more reaction chambers are visible through the reaction card such that a readout indicator within each reaction chamber is visible for indicating results of the assay.

Clause 2. The assay device of clause 1, further comprising an outer cover configured to house the reaction card.

Clause 3. The assay device of clause 2, wherein the outer cover comprises an aperture to enable connection of the buffer tube to the reaction card.

Clause 4. The assay device of clause 2 or 3, wherein the outer cover comprises a lid that is selectively openable and closable and provides visual access to an upper surface of the reaction card.

Clause 5. The assay device of any preceding clause, further comprising a readout card overlaying or lying adjacent to the one or more reaction chambers, the reaction card including one or more reaction chamber labels and optionally a colorimetric results label.

Clause 6. The assay device of any preceding clause, wherein the reaction card further comprises a receiving base configured for receiving the buffer tube in a vertically upright position.

Clause 7. The assay device of any preceding clause, wherein the device is configured to cause release of the contents of the buffer tube upon connection of the buffer tube to the reaction card.

Clause 8. The assay device of clause 7, wherein connection of the buffer tube to the reaction card causes a frangible seal to break.

Clause 9. The assay device of any preceding clause, wherein the reaction card includes a laminate construction.

Clause 10. The assay device of clause 9, wherein the reaction card includes a card top defining the one or more reaction chambers and the one or more channels, the card top optionally being formed from a rigid polymer.

Clause 11. The assay device of clause 10, wherein the reaction card includes a channel backing disposed below the card top and defining a bottom of the one or more channels, the channel backing optionally being formed from a flexible polymer film.

Clause 12. The assay device of clause 10 or 11, wherein the reaction card comprises a chamber bottom disposed below the card top and defining a bottom of the one or more reaction chambers, the chamber bottom comprising apertures and/or vent holes aligned with the overlying one or more reaction chambers, the chamber bottom optionally being formed from a flexible polymer film.

Clause 13. The assay device of clause 12, wherein the reaction card comprises a venting membrane disposed below the chamber bottom and configured to allow escape of gasses from the one or more reaction chambers.

Clause 14. The assay device of any one of clauses 10-13, wherein the reaction card comprises a card bottom defining a bottom of the reaction card, the card bottom optionally being formed from a rigid polymer.

Clause 15. The assay device of any preceding clause, wherein the one or more channels are microfluidic channels configured to draw the contents of the buffer tube toward the one or more reaction chambers via capillary action.

Clause 16. The assay device of any preceding clause, wherein the reaction card comprises a plurality of reaction chambers.

Clause 17. The assay device of clause 16, wherein each reaction chamber is configured to assay a different target nucleic acid.

Clause 18. The assay device of clause 16 or 17, wherein at least one of the plurality of reaction chambers is configured as a positive control.

Clause 19. The assay device of any preceding clause, wherein at least one of the one or more reaction chambers connects to a corresponding channel at a joint configured with a fillet structure, the fillet structure imparting a curve that avoids a 90° angle at the joint.

Clause 20. The assay device of clause 19, wherein an angle between the at least one reaction chamber and the corresponding channel imparted by the fillet structure is about 110° to about 155°, or about 115° to about 145°, or about 120° to about 135°.

Clause 21. The assay device of any preceding clause, wherein the buffer tube comprises a cap portion, a swab portion connected to the cap portion and extending therefrom, and a body portion in which the swab portion is insertable and to which the cap portion is attachable.

Clause 22. The assay device of any preceding clause, further comprising a heat source and a temperature regulator disposed between the heat source and the reaction card.

Clause 23. The assay device of clause 22, wherein the temperature regulator comprises a phase change material, such as one or more solvents, with a boiling point that is at or above a target reaction temperature for the reaction card.

Clause 24. The assay device of clause 23, wherein the temperature regulator comprises a pouch in which the phase change material and an optional solid carrier are disposed.

Clause 25. The assay device of any one of clauses 22-24, wherein the heat source generates heat via an exothermic reaction, such as an oxygen driven exothermic reaction.

Clause 26. The assay device of clause 25, wherein an air inlet provides air to the heat source, and wherein the temperature regulator is expandable such that upon expansion during heating, the temperature regulator restricts the air inlet.

Clause 27. The assay device of clause 26, wherein a first side of the heat source faces the temperature regulator, and wherein the air inlet is disposed on a second side of the heat source, in between the second side of the heat source and an interior surface of the assay device, such as a surface of an insulation layer of the assay device.

Clause 28. The assay device of any one of clauses 22-27, wherein an outer cover comprises multiple sections connected to one another and configured to fold, wherein the outer cover comprises an interior stiffener that folds to a position within an interior of the outer cover to provide structural support.

Clause 29. The assay device of clause 28, wherein the interior stiffener includes a cutout for the heat source and provides structural support that assists in allowing vertical movement of the heat source during operation of the assay device.

Clause 30. The assay device of any preceding clause, wherein the master mix of the one or more reaction chambers are formulated to enable a loop-mediated isothermal amplification (LAMP), or reverse transcription LAMP (RT-LAMP) reaction.

Clause 31. The assay device of any preceding clause, wherein the readout indicator is formulated to be visible by direct naked eye visualization.

Clause 32. A method for assaying a sample for one or more target nucleic acids, the method comprising: using an assay device as in any preceding clause, adding a sample to the sample processing buffer within the buffer tube to form a sample mixture; connecting the buffer tube to the reaction card to cause at least a portion of the sample mixture to migrate to the one or more reaction chambers; and providing heat to the reaction card to drive isothermal amplification of target nucleic acids if such are present within the sample.

Clause 33. The method of clause 32, wherein upon mixing of the sample mixture with the master mix in the one or more reaction chambers, a one-pot reaction is carried out that releases nucleic acids, amplifies target nucleic acids if present in the sample, and activates the readout indicator if target nucleic acids are amplified.

Clause 34. The method of clause 32 or 33, wherein providing heat to the reaction card comprises utilizing a heat source and temperature regulator of the assay device as in any one of claims 22-29.

Clause 35. The method of any one of clauses 32-34, wherein the assay detects the presence of one or more pathogens within the sample, such as one or more of SARS-CoV-2, Flu A, Flu B, or Respiratory Syncytial Virus (RSV).

Clause 36. The method of any one of clauses 32-35, wherein the isothermal amplification comprises loop-mediated isothermal amplification (LAMP), or reverse transcription LAMP (RT-LAMP).

Clause 37. The method of any one of clauses 32-36, wherein the sample is a lower nasal swab sample, nasopharyngeal swab sample, gingival swab sample, buccal swab sample, gargle sample, sputum sample, or saliva sample.

### ADDITIONAL TERMS & DEFINITIONS

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, reagent components, phase change materials, and/or structural features of the assay device that are not specifically described herein may optionally be completely omitted or essentially omitted.

An embodiment that “essentially omits” or is “essentially free of” a component may include trace amounts and/or non-functional amounts of the component. For example, an “essentially omitted” component may be included in an amount no more than 2.5%, no more than 1%, no more than 0.1%, or no more than 0.01% by total weight of the composition. This is likewise applicable to other negative modifier phrases such as, but not limited to, “essentially omits,” “essentially without,” similar phrases using “substantially” or other synonyms of “essentially,” and the like.

A composition that “completely omits” or is “completely free of” a component does not include a detectable amount of the component (i.e., does not include an amount above any inherent background signal associated with the testing instrument) when analyzed using standard coating composition analysis techniques such as, for example, chromatographic techniques (e.g., thin-layer chromatography (TLC), gas chromatography (GC), liquid chromatography (LC)), or spectroscopy techniques (e.g., Fourier transform infrared (FTIR) spectroscopy).

1. An assay device configured for isothermal amplification and detection of nucleic acids, the assay device comprising:
a buffer tube containing a sample processing buffer, the sample processing buffer formulated for mixing with a sample; and
a reaction card configured to receive the buffer tube and at least a portion of its contents, the reaction card comprising
a receiving area for receiving the contents of the buffer tube,
one or more reaction chambers each containing a master mix formulated to enable amplification of a target nucleic acid, and
one or more channels fluidically connecting the receiving area with the one or more reaction chambers and configured to deliver the received contents of the buffer tube to the one or more reaction chambers,
wherein the one or more reaction chambers are visible through the reaction card such that a readout indicator within each reaction chamber is visible for indicating results of the assay.

2. The assay device of claim 1, further comprising a readout card overlaying or lying adjacent to the one or more reaction chambers, the reaction card including one or more reaction chamber labels and optionally a colorimetric results label.

3. The assay device of claim 1, wherein the device is configured to cause release of the contents of the buffer tube upon connection of the buffer tube to the reaction card.

4. The assay device of claim 3, wherein connection of the buffer tube to the reaction card causes a frangible seal to break.

5. The assay device of claim 1, wherein the reaction card includes a card top defining the one or more reaction chambers and the one or more channels.

6. The assay device of claim 5, wherein the reaction card comprises a chamber bottom disposed below the card top and defining a bottom of the one or more reaction chambers, the chamber bottom comprising apertures and/or vent holes aligned with the overlying one or more reaction chambers.

7. The assay device of claim 6, wherein the reaction card comprises a venting membrane disposed below the chamber bottom and configured to allow escape of gasses from the one or more reaction chambers.

8. The assay device of claim 1, wherein the one or more channels are microfluidic channels configured to draw the contents of the buffer tube toward the one or more reaction chambers via capillary action.

9. The assay device of claim 1, wherein the reaction card comprises a plurality of reaction chambers.

10. The assay device of claim 9, wherein each reaction chamber is configured to assay a different target nucleic acid.

11. The assay device of claim 1, wherein at least one of the one or more reaction chambers connects to a corresponding channel at a joint configured with a fillet structure, the fillet structure imparting a curve that avoids a 90° angle at the joint.

12. The assay device of claim 11, wherein an angle between the at least one reaction chamber and the corresponding channel imparted by the fillet structure is about 110° to about 155°.

13. The assay device of claim 1, further comprising a heat source and a temperature regulator disposed between the heat source and the reaction card.

14. The assay device of claim 13, wherein the temperature regulator comprises a phase change material with a boiling point that is at or above a target reaction temperature for the reaction card.

15. The assay device of claim 13, wherein the heat source generates heat via an oxygen driven exothermic reaction.

16. The assay device of claim 15, wherein an air inlet provides air to the heat source, and wherein the temperature regulator is expandable such that upon expansion during heating, the temperature regulator restricts the air inlet.

17. The assay device of claim 16, wherein a first side of the heat source faces the temperature regulator, and wherein the air inlet is disposed on a second side of the heat source, in between the second side of the heat source and an interior surface of the assay device, such as a surface of an insulation layer of the assay device.

18. The assay device of claim 1, wherein the master mix of the one or more reaction chambers are formulated to enable a loop-mediated isothermal amplification (LAMP) or reverse transcription LAMP (RT-LAMP) reaction.

19. The assay device of claim 1, wherein the readout indicator is formulated to be visible by direct naked eye visualization.

20. A method for assaying a sample for one or more target nucleic acids, the method comprising:
using an assay device as in claim 1, adding a sample to the sample processing buffer within the buffer tube to form a sample mixture;
connecting the buffer tube to the reaction card to cause at least a portion of the sample mixture to migrate to the one or more reaction chambers; and
providing heat to the reaction card to drive isothermal amplification of target nucleic acids if such are present within the sample.
